(19) United States
(12) Patent Application Publication
(10) Pub. No.: US 2025/0312063 A1
(43) Pub. Date: Oct. 9, 2025

(54) INTRAOSSEOUS NEEDLE

(71) Applicant: Regents of the University of Minnesota, Minneapolis, MN (US)

(72) Inventors: Dilshan Rajan, Minneapolis, MN (US); Anuk Dias, Maple Grove, MN (US); Shaliny Jadhav, Maple Grove, MN (US)

(21) Appl. No.: 19/097,128

(22) Filed: Apr. 1, 2025

Related U.S. Application Data
(60) Provisional application No. 63/575,443, filed on Apr. 5, 2024.

(51) Int. Cl. A61B 17/34

(52) U.S. Cl. CPC A61B 17/3472; A61B 17/3496; A61B 2017/3433; A61B 2017/3458

(57) ABSTRACT

An intraosseous needle hub includes a hub body and a needle. The hub body has a hollow bore extending along a central axis from a proximal end to a distal end of the hub body. The needle has a hollow bore extending along the central axis and includes a proximal end attached to the hub body, a distal end including a needle tip, a threaded section at the needle tip configured to screw into bone of a patient, and a shoulder portion between the threaded section and the hub body. The shoulder portion is configured to resist movement of the needle through bone of a patient.

### CROSS REFERENCE TO RELATED APPLICATION

The present application is based on and claims the benefit of U.S. provisional patent application Ser. No. 63/575,443, filed Apr. 5, 2024, the content of which is hereby incorporated by reference in its entirety.

### FIELD

Embodiments of the present disclosure generally relate to an intraosseous needle and, more particularly, to an improved intraosseous needle hub that simplifies and improves the safety of gaining vascular access through a bone of a patient.

### BACKGROUND

Intraosseous (IO) needles are an essential tool used in emergency medical situations when the need for immediate vascular access is paramount and other conventional routes are not accessible. This is usually seen in the pediatric population, especially ages 12 and under, where gaining access intravenously is difficult, but is also being used more frequently in adults as well. In these emergencies, an intraosseous needle is used to drill through the cortical bone (e.g., anteromedial proximal tibia) of the patient and into the marrow cavity, or the intraosseous space, to gain vascular access.

IO needles generally comprise a stylet component and needle hub. The stylet component includes a stylet that is attached to a stylet body. The needle hub includes a hub body and a needle having a hollow bore. The stylet is inserted through a bore in the hub body and the bore of the needle and the stylet body is screwed to the hub body to form an assembly. The tip of the stylet is drilled through the cortical bone of the patient such that the tip of the needle is within the intraosseous space. The stylet component is then screwed off the needle hub and removed, allowing for direct vascular access through the bore of the needle hub.

Conventional IO needle designs have two major problems. One problem is with infiltration (in the extreme case, extravasation) where the needle goes completely through the bone and out the other side causing fluid to leak into the surrounding tissue. This issue can lead to inflammatory reactions, compartment syndrome, tissue necrosis, and skin breakdown. Some studies have found this issue has an occurrence of nearly 12 percent.

Conventional IO needle designs also have issues with needle displacement. During and after the conventional needle is inserted into a bone of the patient, the needle frequently shifts or moves out of the body, wasting critical time and potentially causing additional problems such as compartment syndrome.

### SUMMARY

Embodiments of the present disclosure include an intraosseous (IO) needle assembly, a needle hub of the IO assembly, and methods of tapping an interosseous space within a bone of a patient using the IO assembly.

One example of the intraosseous needle hub includes a hub body and a needle. The hub body has a hollow bore extending along a central axis from a proximal end to a distal end of the hub body. The needle has a hollow bore extending along the central axis and includes a proximal end attached to the hub body, a distal end including a needle tip, a threaded section at the needle tip configured to screw into bone of a patient, and a shoulder portion between the threaded section and the hub body. The shoulder portion is configured to resist movement of the needle through bone of a patient.

One example of the intraosseous needle assembly includes a needle hub, and a stylet component. The needle hub includes a hub body having a hollow bore extending along a central axis from a proximal end to a distal end of the hub body, and a needle having a hollow bore extending along the central axis. The needle includes a proximal end attached to the hub body, a distal end including a needle tip, a threaded section at the needle tip configured to screw into bone of a patient, and a shoulder portion between the threaded section and the hub body. The shoulder portion is configured to resist movement of the needle through bone of a patient. The stylet component includes a stylet body and a stylet. The stylet body has a proximal end and a distal end, which is detachably connected to the proximal end of the needle hub. The stylet is attached to the distal end of the stylet body and extends along the central axis through the hollow bores of the hub body and the needle.

In an example method of tapping an intraosseous space within a bone of a patient using an intraosseous needle assembly, the intraosseous needle assembly includes a needle hub and a stylet component. The needle hup includes a hub body having a hollow bore extending along a central axis from a proximal end to a distal end of the hub body, and a needle having a hollow bore extending along the central axis. The needle including a proximal end attached to the hub body, a distal end including a needle tip, a threaded section at the needle tip and a shoulder portion between the threaded section and the hub body. The shoulder portion is configured to resist movement of the needle through bone of a patient. The stylet component includes a stylet body having a proximal end and a distal end, which is detachably connected to the proximal end of the needle hub, and a stylet attached to the distal end of the stylet body and extending along the central axis through the hollow bores of the hub body and the needle. The method includes screwing the needle tip and the threaded section into the bone until the shoulder portion engages the bone and detaching the stylet component from the needle hub.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIG. 1 is an isometric view of an example IO needle assembly, in accordance with embodiments of the present disclosure.

FIG. 2 is a cross-sectional view of the example IO needle assembly, in accordance with embodiments of the present disclosure.

FIG. 3 is an exploded isometric view of the example IO needle assembly, in accordance with embodiments of the present disclosure.

FIG. 4 is an isometric view of an example of a stylet component, in accordance with embodiments of the present disclosure.

FIG. 5 is a cross-sectional view of an example of a needle hub, in accordance with embodiments of the present disclosure.

FIG. 6 is a side view of a distal end of an example needle, in accordance with embodiments of the present disclosure.

FIG. 7 is a simplified diagram of the distal end of the example needle viewed along a central axis, in accordance with embodiments of the present disclosure.

FIGS. 8A-C are simplified cross-sectional diagrams illustrating a method of using the intraosseous assembly to tap an intraosseous space within a bone of a patient, in accordance with embodiments of the present disclosure.

### DETAILED DESCRIPTION OF ILLUSTRATIVE EMBODIMENTS

Embodiments of the present disclosure are described more fully hereinafter with reference to the accompanying drawings. Elements that are identified using the same or similar reference characters refer to the same or similar elements. The various embodiments of the present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art.

Embodiments of the present disclosure include an intraosseous (IO) needle assembly, a needle hub of the IO needle assembly, and methods of tapping an interosseous space within a bone of a patient using the IO needle assembly. The IO needle assembly and needle hub provide advantages over conventional IO needle assemblies and needle hubs including, for example, features that limit and preset the distance the needle of the needle hub may be inserted through the cortical bone of a patient to prevent under-insertion and over-insertion of the needle and ensuring proper positioning of the needle tip in the desired intraosseous space. Additionally, the needle of the needle hub is configured to securely attach to the cortical bone of the patient and reduce the likelihood of needle displacement.

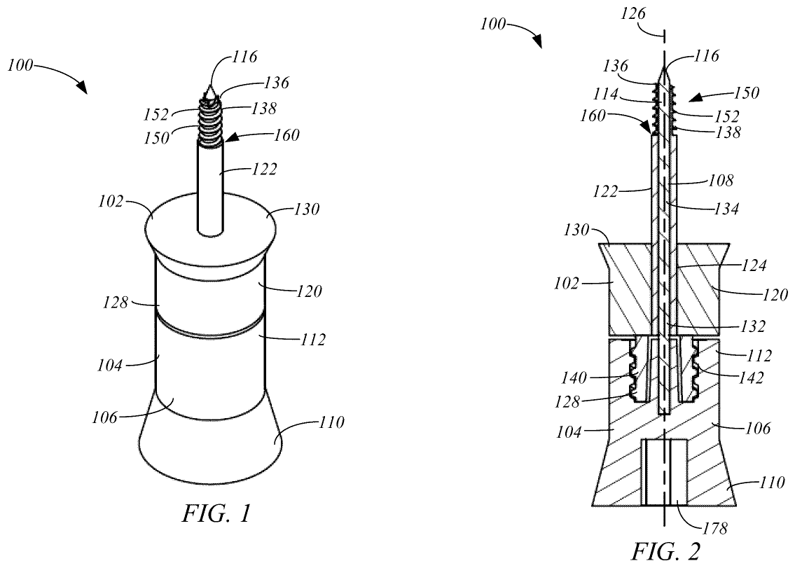

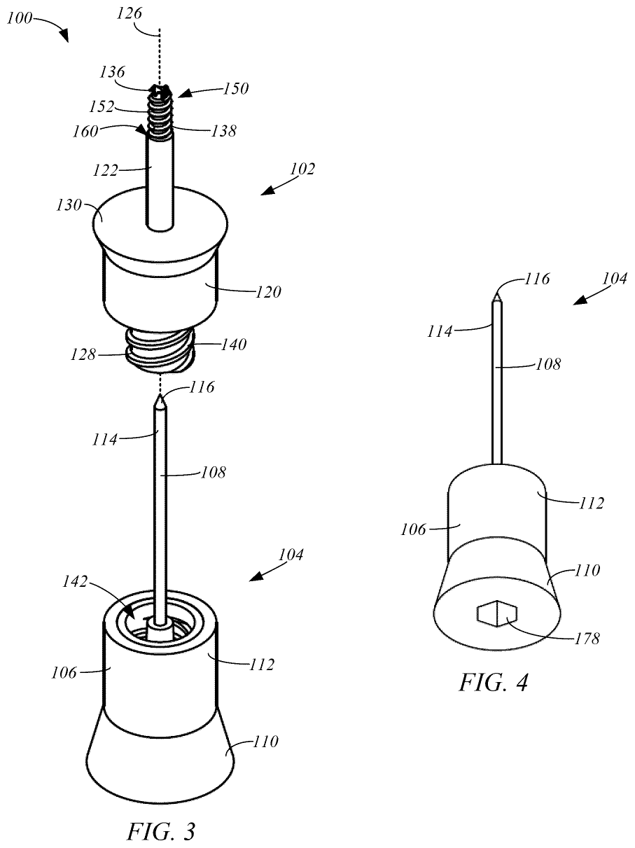

An example IO needle assembly 100 in accordance with embodiments of the present disclosure is shown in FIGS. 1-3. FIG. 1 is an isometric view of the IO needle assembly 100, FIG. 2 is a cross-sectional view of the IO needle assembly 100, and FIG. 3 is an exploded isometric view of the IO needle assembly 100.

The IO needle assembly 100 includes a needle hub 102 and a stylet component 104. FIG. 4 is an isometric view of an example of the stylet component 104 and FIG. 5 is a cross-sectional view of an example of the needle hub 102, in accordance with embodiments of the present disclosure.

The stylet component 104 includes a stylet body 106 and a stylet 108. The stylet body 106 has a proximal end 110 and a distal end 112. The stylet 108 is attached to the distal end 112 of the stylet body 106. A distal end 114 of the stylet 108 may include a pointed tip 116.

The needle hub 102 includes a hub body 120 and a needle 122. The hub body 120 has a hollow bore 124 extending along a central axis 126 from a proximal end 128 to a distal end 130 of the hub body 120. A proximal end 132 of the needle 122 is attached to the hub body 120 and may be received within the bore 124 of the hub body 120. The needle has a hollow bore 134 through which the central axis 126 extends. The needle 122 includes a needle tip 136 at a distal end 138. In some embodiments, the needle tip 136 is fluted to facilitate drilling into cortical bone.

The stylet body 106 is configured to attach to the hub body 120 using conventional techniques to form the assembly 100, such that the stylet 108 extends through the bores 124 and 134 and the stylet tip 116 extends through the needle tip 136, as shown in FIG. 2. For example, the hub body 120 may include a connector 140, such as a threaded screw (FIGS. 2, 3 and 5), at the proximal end 128 that is configured cooperate with a connector 142 of the stylet body 106, such as a threaded socket (FIGS. 2 and 3), that is accessible at the distal end 112 of the stylet body 106 to secure the hub body 120 to the stylet body 106. In one embodiment, the connector 140 comprises a conventional Luer lock fitting that is configured to connect an intravenous fluid line to the bore 134 of the needle 122.

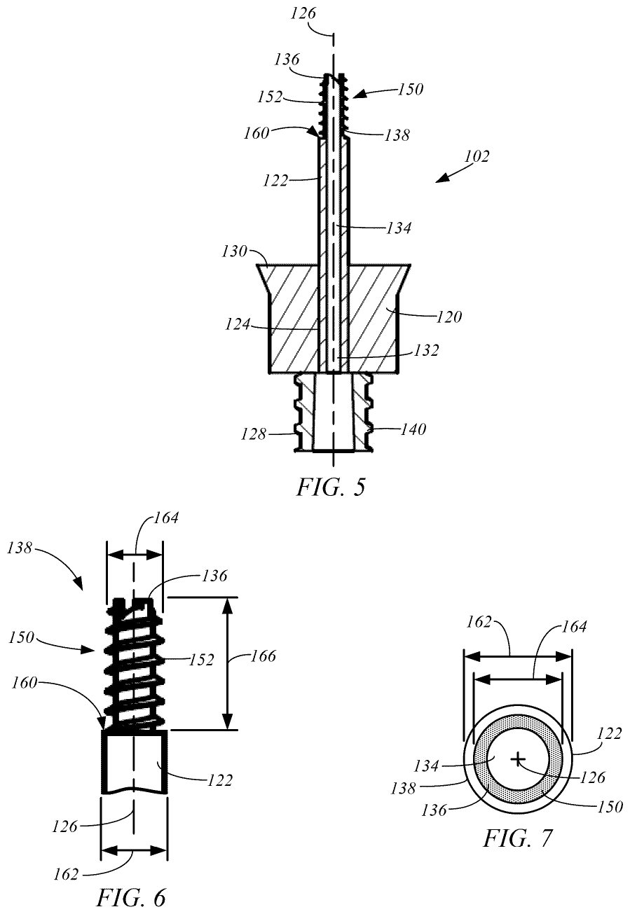

FIG. 6, which is a side view of the distal end 138 of the example needle 122, in accordance with embodiments of the present disclosure. In some embodiments, the needle 122 includes a threaded section 150 at the needle tip 136 comprising screw threads 152 that are configured to screw into bone of a patient in response to rotation of the needle 122 or the assembly 100 about the central axis 126. In some embodiments, the threaded section 150 is self-tapping (e.g., thread cutting or thread forming). The threaded section 150 may have a length measured along the central axis 126 of greater than about 2.0 millimeters, about 2.0-10.0 millimeters, or about 4.0-6.0 millimeters, for example.

In some embodiments, the needle 122 includes a shoulder portion 160 that is located between the threaded section 150 and the hub body 120. The shoulder portion 160 may adjoin the threaded section 150, as shown in FIG. 6, or it may be displaced from the threaded section 150 toward the hub body 120, for example.

The shoulder portion 160 generally operates to limit or inhibit a distance the distal end 138 of the needle 122 and the threaded section 150 may be screwed into cortical bone of a patient. For example, as the threaded section 150 is screwed into bone of a patient it moves along the central axis 126 relative to the bone until the shoulder portion 160 engages the bone, at which point further movement of the threaded section 150 and needle tip 136 along the central axis 126 relative to the bone is prevented or resisted by the shoulder portion 160. The blockage or resistance to further movement of the needle 122 may be sensed by the physician and used to trigger an end to the screwing process. As a result, the shoulder portion 160 assists in setting the distance the needle tip 136 extends into a bone.

In some embodiments, the shoulder portion 160 extends radially from the central axis 126 a distance that is greater than or equal to that of the threaded section 150. For example, the shoulder portion 160 may be an annular shoulder having a diameter 162 that is greater than or equal to a diameter 164 of the threaded section 150, as indicated in FIG. 6 and FIG. 7, which is a simplified diagram of the distal end 138 of the needle 122 viewed along the central axis 126, in accordance with embodiments of the present disclosure. Alternatively, the shoulder portion 160 may comprise one or more tabs that extend radially from the central axis 126.

A distance 166 the shoulder portion is displaced from the needle tip, which may correspond to the length of the threaded section, is selected to ensure that the distal tip 136 is located within the intraosseous space of a selected bone. In some embodiments, the distance 166 is selected to be slightly larger (e.g., 10-25% larger) than an anticipated maximum thickness of the cortical bone layer of a selected bone that is to be penetrated by the needle 122. For example, the maximum thickness of the cortical bone layer of a tibia of a pediatric patient may be approximately 4.4-4.8 millimeters, such as around 4.6 millimeters. Thus, the distance 166 may be set to about 5.0-6.0 millimeters, such as 5.5 millimeters, for example, for use with the tibia of pediatric patients, for example. Other distances 166 may be selected to accommodate different bones, adult patients, infant patients, etc.

Thus, the distance 166 that the shoulder portion 160 is located from the needle tip 136 places a limit on the distance that that the needle tip 136 may be inserted into a bone and prevents over-insertion of the needle tip 136 into the bone. Additionally, the distance 166 also ensures that the needle tip is not under-inserted into the bone and properly extends through the cortical bone layer into the intraosseous space. Thus, the shoulder portion 160 simplifies the positioning of the needle tip 136 with the intraosseous space of a bone and avoids the over and under insertion problems of conventional intraosseous needles.

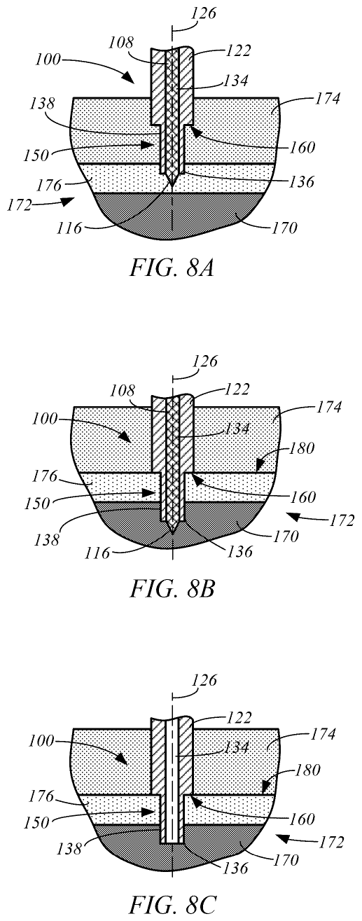

FIGS. 8A-C are simplified cross-sectional diagrams illustrating a method of using the intraosseous needle assembly 100 to tap an intraosseous space 170 within a bone 172 of a patient, in accordance with embodiments of the present disclosure. After forming the IO needle assembly 100, the tip 136 of the needle 122, the threaded section 150 and the tip 116 of the stylet 108 are screwed or drilled through soft tissue 174 of the patient and into a cortical bone layer 176, such as that of the tibia, for example. This may be facilitated using a suitable drill having a drill bit (e.g., hex bit) that is received within a socket 178 at the proximal end 110 of the stylet body 106, such as that shown in FIG. 4.

The screwing of the assembly 110 drives the needle tip 136 along the central axis 126 relative to the cortical bone layer 176 until the shoulder portion 160 engages the surface 180 of the cortical bone layer 176, as shown in FIG. 8B. At this point, further movement of the needle tip 136 along the central axis 126 relative to the cortical bone layer 176 is prevented or inhibited by the shoulder portion 160. The distance 166 (FIG. 6) separating the shoulder portion 160 from the needle tip 136 ensures that the needle tip is positioned within the intraosseous space 170 of the bone 172, as shown in FIG. 8B.

The physician may then detach the stylet body 106 from the hub body 120 to remove the stylet component 104 from the assembly 110, which leaves a pathway to the intraosseous space through the bore 134 of the needle 122, as shown in FIG. 8C. An intravenous fluid line may then be connected to the connector 140 (e.g., Luer lock fitting) of the hub body 120 and used to provide an intravenous treatment to the patient, such as the delivery of fluids into the intraosseous space 170, through the bore 134.

The threaded section 150 secures the distal end 138 of the needle 122 to the cortical bone layer 176 and prevents its displacement during normal operations, such as during the removal of the stylet component 104, the connection of the intravenous fluid line to the hub body 120, and normal contact with the hub body 120, for example. Thus, the needle hub 102 avoids the displacement issues of conventional needle hubs.

In some embodiments, the shoulder portion 160 and the threaded section 150 also facilitate the formation of a seal between the surface 180 of the cortical bone layer 176 and the needle 122. This assists in preventing undesired leakage from the intraosseous space 170.

Although the embodiments of the present disclosure have been described with reference to preferred embodiments, workers skilled in the art will recognize that changes may be made in form and detail without departing from the spirit and scope of the present disclosure.

What is claimed is:

1. An intraosseous needle hub comprising:
a hub body having a hollow bore extending along a central axis from a proximal end to a distal end of the hub body; and
a needle having a hollow bore extending along the central axis and comprising:
a proximal end attached to the hub body;
a distal end including a needle tip;
a threaded section at the needle tip configured to screw into bone of a patient; and
a shoulder portion between the threaded section and the hub body, the shoulder portion configured to resist movement of the needle through bone of a patient.

2. The intraosseous needle hub according to claim 1, wherein the threaded section extends a length of greater than about 2.0 millimeters along the central axis from the needle tip.

3. The intraosseous needle hub according to claim 1, wherein the needle tip is fluted and/or the threaded section is self-tapping.

4. The intraosseous needle hub according to claim 1, wherein the shoulder portion extends radially from the central axis a greater distance than the threaded section.

5. The intraosseous needle hub according to claim 4, wherein the shoulder portion comprises an annular shoulder that extends around the central axis and is displaced along the central axis about 5.0-6.0 millimeters from the needle tip.

6. The intraosseous needle hub according to claim 5, including a connector attached to the proximal end of the hub body.

7. The intraosseous needle hub according to claim 6, wherein the connector comprises a Luer lock fitting.

8. An intraosseous needle assembly comprising:
a needle hub comprising:
a hub body having a hollow bore extending along a central axis from a proximal end to a distal end of the hub body; and
a needle having a hollow bore extending along the central axis and comprising:
a proximal end attached to the hub body;
a distal end including a needle tip;
a threaded section at the needle tip configured to screw into bone of a patient; and
a shoulder portion between the threaded section and the hub body, the shoulder portion configured to resist movement of the needle through bone of a patient; and
a stylet component comprising:
a stylet body having a proximal end and a distal end, which is detachably connected to the proximal end of the needle hub; and
a stylet attached to the distal end of the stylet body and extending along the central axis through the hollow bores of the hub body and the needle.

9. The intraosseous needle assembly according to claim 8, wherein the threaded section extends a length of greater than about 2.0 millimeters along the central axis from the needle tip.

10. The intraosseous needle assembly according to claim 8, wherein the needle tip is fluted and/or the threaded section is self-tapping.

11. The intraosseous needle assembly according to claim 8, wherein the shoulder portion extends radially from the central axis a greater distance than the threaded section.

12. The intraosseous needle assembly according to claim 11, wherein the shoulder portion comprises an annular shoulder that extends around the central axis and is displaced along the central axis about 5.0-6.0 millimeters from the needle tip.

13. The intraosseous needle assembly according to claim 8, wherein:
the proximal end of the hub body includes a first connector; and
the distal end of the stylet body includes a second connector that is configured to detachably connect to the first connector.

14. The intraosseous needle assembly according to claim 13, wherein the first connector comprises a Luer lock fitting.

15. A method of tapping an intraosseous space within a bone of a patient using an intraosseous needle assembly, which includes:
a needle hub comprising:
a hub body having a hollow bore extending along a central axis from a proximal end to a distal end of the hub body; and
a needle having a hollow bore extending along the central axis and comprising:
a proximal end attached to the hub body;
a distal end including a needle tip;
a threaded section at the needle tip; and
a shoulder portion between the threaded section and the hub body, the shoulder portion configured to resist movement of the needle through bone of a patient; and
a stylet component comprising:
a stylet body having a proximal end and a distal end, which is detachably connected to the proximal end of the needle hub; and
a stylet attached to the distal end of the stylet body and extending along the central axis through the hollow bores of the hub body and the needle,
the method comprising:
screwing the needle tip and the threaded section into the bone until the shoulder portion engages the bone; and
detaching the stylet component from the needle hub.

16. The method according to claim 15, including forming a seal between the bone and the needle using the shoulder portion.

17. The method according to claim 15, including delivering fluids into the intraosseous space through the hollow bore of the needle.

18. The method according to claim 15, wherein:
extends radially from the central axis a greater distance than the threaded section;
the shoulder portion is displaced along the central axis about 5.0-6.0 millimeters from the needle tip; and/or
the shoulder portion comprises an annular shoulder that extends around the central axis and has a diameter that is greater than a diameter of the threaded section.

19. The method according to claim 15, wherein:
the threaded section extends a length of greater than about 2.0 millimeters along the central axis from the needle tip;
the needle tip is fluted; and/or
the threaded section is self-tapping.

20. The method according to claim 15, wherein:
the proximal end of the hub body includes a first connector; and
the distal end of the stylet body includes a second connector that is configured to detachably connect to the first connector.
